(19) United States
(12) Patent Application Publication
(10) Pub. No.: US 2025/0161694 A1
(43) Pub. Date: May 22, 2025

(54) HUMAN-POWERED STIMULATING/SENSING IMPLANT SYSTEM

(71) Applicant: Evoke Medical, LLC dba DirectSync Surgical, Lawrence, KS (US)

(72) Inventors: Leighton LaPierre, Hampstead, NC (US); Ember Krech, Lawrence, KS (US); Elizabeth Friis, Lawrence, KS (US); Paul Arnold, Champaign, IL (US); Zygmunt Porada, Wayne, PA (US); Michael Bradley, Linden, PA (US); Safakcan Tuncdemir, Linden, PA (US); Mario Lopes, Linden, PA (US)

(21) Appl. No.: 18/951,376

(22) Filed: Nov. 18, 2024

Related U.S. Application Data
(60) Provisional application No. 63/600,002, filed on Nov. 16, 2023.

(51) Int. Cl. A61N 1/378; A61B 5/0538; A61B 5/11; A61N 1/372

(52) U.S. Cl. CPC A61N 1/3785; A61B 5/0538; A61B 5/112; A61N 1/37223; A61B 2560/0214

(57) ABSTRACT

An implant assembly includes a housing configured to store components that generate mechanically synced electrical stimulation, a first electrode positioned on an inferior surface of the housing, and a second electrode positioned on a superior surface of the housing, the components including a force activated piezogenerator electrically connected to a impedance matched rectifying circuit, wherein the piezogenerator transduces a mechanical load to an electrical signal that is passed through the impedance matched rectifying circuit and delivered through the first and second electrode to a healing site when an anatomical force is applied to the first and second electrode that make contact with two bony surfaces.

### CROSS-REFERENCE TO RELATED APPLICATION

The present application claims priority benefit of U.S. Provisional Application No. 63/600,002, filed Nov. 16, 2023, which is herein incorporated by reference in its entirety.

### GOVERNMENT LICENSE RIGHTS TO CONTRACTOR-OWNED INVENTIONS MADE UNDER FEDERALLY SPONSORED RESEARCH AND DEVELOPMENT

This invention was made with government support under R44 AR070088 and R43 AR082237 awarded by the National Institutes of Health. The government has certain rights in the invention.

### BACKGROUND OF THE INVENTION

In general the present invention relates to implantable devices, and more particularly to a human-powered stimulating/sensing implant system.

In general, electrical stimulation of body tissues is used throughout medicine for treatment of both chronic and acute conditions. One such therapeutic application is using electrical stimulation to increase the rate of bone regrowth, repair, fusion of bones or bone grafts. For example, Electrical bone growth stimulation (EBGS) is the treatment of bone fusion or repair using electrical current (direct current or alternating current). Currently, invasive use of these devices involves surgical implantation of a current generator in an intramuscular or subcutaneous space, while an electrode is implanted within the fragments of bone or bone graft at the bone fusion site.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In an aspect, the present invention features an implant assembly including a housing configured to store components that generate mechanically synced electrical stimulation, a first electrode positioned on an inferior surface of the housing, and a second electrode positioned on a superior surface of the housing, the components including a force activated piezogenerator electrically connected to a impedance matched rectifying circuit, wherein the piezogenerator transduces a mechanical load to an electrical signal that is passed through the impedance matched rectifying circuit and delivered through the first and second electrode to a healing site when an anatomical force is applied to the first and second electrode that make contact with two bony surfaces.

In another aspect, the present invention features a human powered implantable device including a first electrode positioned on an exterior surface of a housing, a second electrode positioned on an exterior surface of the housing and isolated from the first electrode, a microcontroller, and piezoelectric materials within the housing to generate load-induced power and run a measurement and storage function, the power configured to generate electrical stimulation to promote bone growth and support data collection related to load, impedance, acceleration, temperature, and other physiologic signals.

In still another aspect, the invention features a system including an external circuit, and an implant, the implant configured to wirelessly communicate with the external circuit, the implant including a piezo construct, an analog circuit, the piezo construct linked to the analog circuit, a digital circuit, electrodes, the electrodes linked to the analog circuit and the digital circuit, a solid state battery, the solid state battery linked to the digital circuit, a near field communication (NFC) antenna, and a Bluetooth Low Energy (BLE) antenna, the NFC antenna and the BLE antenna linked to the digital circuit.

These and other advantages of the invention will be further understood and appreciated by those skilled in the art by reference to the following written specification, claims and appended drawings.

### BRIEF DESCRIPTION OF DRAWINGS

FIG. 1 illustrates a block diagram.

FIG. 2 illustrates an exemplary implant and a graph.

FIG. 3 illustrates an exemplary biological and electrical feedback loop.

FIGS. 4A, 4B, 4C and 4D illustrate load sharing.

FIG. 5. illustrates a graph.

FIG. 6 illustrates operational and nonoperational regions.

FIG. 7 illustrates a graph.

FIG. 8 illustrates an exemplary preferred embodiment.

FIG. 9 illustrates an exemplary preferred embodiment.

FIG. 10 illustrates an exploded view of the preferred embodiment.

FIG. 11 illustrates an exemplary circuit-construct sub assembly.

FIG. 12 illustrates a PEEK body component.

FIG. 13 illustrates an electrode endplate component.

FIG. 14 is a block diagram.

### DETAILED DESCRIPTION OF THE PREFERRED EMBODIMENT(S)

It is to be understood that the specific devices and processes illustrated in the attached drawings and described in the following specification are exemplary embodiments of the inventive concepts defined in the appended claims. Hence, specific dimensions and other physical characteristics relating to the embodiments disclosed herein are not to be considered as limiting, unless the claims expressly state otherwise.

The human powered implantable device of the present invention utilizes piezoelectric materials to generate load-induced power and run a diagnostic suite. In addition to electrical stimulation to promote bone growth, the power generated from the human powered implantable device of the present invention supports both directly powering a digital circuit and/or charging a solid state battery in the implant for data collection related to load, impedance, motion, acceleration, temperature, and so forth. One goal for the human-powered implantable device of the present invention is to increase the success of healing and decrease time to fusion via mechanically synced electrical stimulation at the fusion level, as well as give patients and clinicians quantitative measures via onboard sensor technology. The latter provides a way to intervene without a need for expensive CT scans and relying fully on patient self-reporting. Both the sensing and stimulating features of the spinal fusion implant of the present invention give surgeons a way to improve patient outcomes while reducing healthcare costs and generating granular post-operative healing monitoring.

An encapsulated stimulating circuit with additional sensing circuit components is powered by a piezogenerator and dynamic human loading. The implant utilizes power generated from patient motion to deliver bone growth stimulation via mechanically synced electrical stimulation (MSES). A measurement and storage function of the device of the present invention can be activated to collect and store data during discrete periods of time for later transmission to an external data acquisition system.

The sensing capability of the human powered implantable device of the present invention may not be constantly “on” while the patient ambulates. Once the implant is surgically placed and the patient recovers, as the patient ambulates the stimulation is delivered right away—with each step the patient takes. Once power is generated via the piezogenerator and is stimulating for a finite period of time, a switch is triggered to start collecting and storing data for the next X seconds depending on time required to power up the microcontroller and collect data. Alternatively, the sensing mode of the circuit may activate independently at set periodic times of the day or when motion in the patient is sensed. This can also be reversed and that as soon as the device is powered enough for ambulation to start collecting data it does so and then switches to stimulation mode. In one embodiment, every time the implant delivers stimulation for that sustained period of time, it also collects and stores data points for the stimulation session to measure the levels of stimulation being delivered. Potential data types to be collected are, for example, AC voltage from the piezogenerator, local temperature from the microcontroller, impedance between the electrodes, and acceleration for gait analysis.

Because data collection and subsequent storage only occurs for a discrete episode, the stimulation and sensing don't need to be active at the same time. Once the data collection episode is complete, the stimulation resumes. In an ideal scenario, there are 5 to 10 episodes of data collection per day, with each collection tagged with basic metadata, such as date and time. The measurements are transmitted to an external device where trends of activity can be monitored, including prediction of post-operative fusion mass formation, device loosening, biological activity to indicate possible infection and trending. A key value is that the device is able to collect and store data without the patient knowing so there are very little patient compliance issues. The data is stored at frequent intervals but does not need to be high fidelity.

The data transmission requires an external piece of hardware as either an external power source transmitter/receiver to be worn around the waist band and Center placed over the implant or an external home based receiver unit to received low power signals from the implant using protocols such as Bluetooth Low Energy (BLE). This edge device has its own rechargeable or constant power source and connectivity to a mobile app or cloud based database with a GUI. This powers communication to the implant and provides enough power to control and change settings such as stimulation magnitudes and waveforms on the implant device, transfer the stored data from the implant, send it to a mobile device or database in the cloud, the mobile device/app then processing the data and presenting the processed data in a useful manner to both the patient and the clinician.

The implantable device of the present invention collects low fidelity data over time when the device is being biomechanically activated absent of the external transmitter/receiver. This is the majority of the time where the device collects the best max/medium/min when certain power criteria are met. The data read frequency is fairly low in this scenario to reduce power requirements, and so forth. When this data is transmitted to the external edge device and subsequently to cloud database or an app on a smartphone, it can be synchronized with data from other commercially available wearable devices, such as a smartwatch. The combination/synchronization of frequent readily available wearable activity data and internal implant data can produce a data combination that has never been available before.

As mentioned above, the patient uses a mobile app in combination with an external hardware power transfer/data receiver belt to link the implant and to enable two things to happen. First, the implant is fully powered and the low-powered data that has been collected and stored is transferred to the external transmitter/receiver. Second, that transmitter/receiver then switches the implant into a high power sensing mode. This enables other higher power consumption elements, such as an accelerometer, stimulation waveforms to fight infection or other sensors that inform the health status of the patient, or data transfer capabilities to the external receiver to become activated. In this mode, the patient may be instructed to walk a specific distance/time or do different exercises such as bending maneuvers. The external transmitter/receiver belt has its own sensors, such as accelerometers, temperature sensors, and so forth, to correlate and be overlapped with the data from the implant sensors. When the implant is wirelessly powered externally, it can record higher fidelity data at higher frequencies. When the external power mode is shut off or removed from proximity, the implant defaults back to patient powered or self-powered mode for stimulation/low power sensing capabilities. The high power mode in the presence of an external transmitter/receiver can also control the different settings and the function of the microcontroller inside the implant. This enables the bone growth stimulation dosing to be changed or altered depending on the progress of healing. In addition, the high-powered mode can power the device to provide therapeutic stimulation without the patient moving. This enables the physician to prescribe set stimulation dosing and amounts depending on the needs of the patient or for a patient that is less active or mobile and unable to activate the stimulation through human motion.

Data analysis includes leveraging the database from the patient's Electronic Medical Record and preclinical and clinical product studies to process the data and correlate clinical outcomes in order to inform changes to treatment guidelines and the current standard of care. A goal of data acquisition is to collect/display patient data over time. For example, to analyze the changes in the fusion environment, a parameter that has been developed is called relative load change. This parameter relates the voltage measured and stored in the sensor circuit to the change in load experience by the implant in situ.

Similarly, local temperature can be measured and stored during each episode. Once transmitted, trends over time can indicate any potential local infections and or any potential that the device is producing too much heat during stimulation and causing patient harm. Impedance measures over time can indicate biological tissue change and fusion health. These measures ensure that the stimulation is functioning as expected.

Ultimately, data from the sensor in the interbody implant can be clinically studied and validated against existing outcome measures and a standardized clinical determination of fusion correlated via use of a app. Voltage data and other Trends from the implant can be transmitted to the app or cloud database, and the app relating the implant load as compared to the post-op baseline load specific to each patient. As the bony fusion mass forms across the interbody space, load on the implant decreases, thus allowing an assessment of fusion in real time.

In summary, the human powered smart implant of the present invention provides bone (tissue) healing and infection control electrical stimulation activated by human motion and regulated by the changing load environment on the implant, mimicking natural healing progression. As a sustained, dynamic anatomical force is applied to the healing site (e.g., when a patient ambulates), that sustained, dynamic load is transferred to the implant and subsequent electrical stimulation is generated and delivered in sync with the dynamic mechanical load. The rectified and impedance matched direct current electrical stimulation is only delivered when the implant experiences dynamic loading.

The power source and necessary circuitry for the mechanically synced electrical stimulation (MSES) is integrated inside the implant system. In gap healing situations, the MSES can enhance healing in spinal fusion procedures and other orthopedic implant procedures. This concept can be applied to any orthopedic implant where a dynamic mechanical load traverses a cross section of the implant and the piezoelectric material is in line with the mechanical load direction.

The biomechanically activated or load induced or human powered energy is used for three primary reasons, i.e., electrical stimulation (primarily bone growth but could have other benefits such as anti-infection), powering a digital microcontroller to measure and store load/voltage data from the piezo electric material, and re-charging a solid state battery in the implant that could then additionally power electrical stimulation and/or power the digital microcontroller.

In embodiments, the electrodes may not be the endplates.

It should be noted that the sensing functions are not dependent on the stimulation functions. In other words, embodiments can be human powered stimulating and sensing, human powered sensing only and human powered stimulating only.

Using a housing to seal and house the piezo and circuit and then assembled into other implant components may or may not serve as the electrodes.

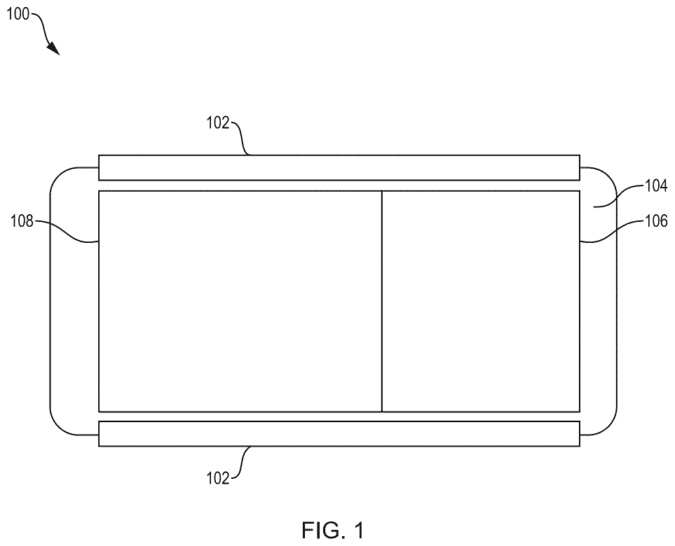

Referring now to FIG. 1, a cross section of an embodiment of an exemplary implant assembly 100 is illustrated. Components that generate mechanically synced electrical stimulation are embedded within a sealed housing 104. In the preferred embodiment, these components are a force activated piezogenerator 106 electrically connected to a impedance matched rectifying circuit and digital microcontroller sensing circuit 108. As the implant 100 experiences a dynamic load due to an anatomical force applied to the superior and inferior surfaces 102 that make contact with two bony surfaces, the piezogenerator 106 transduces the mechanical load to an electrical signal that is passed through the circuit 108 and delivered through the external electrodes 102 to a healing site. In the preferred embodiment, the electrodes 102 are on the inferior and superior surface of the housing 104. In other embodiments, the electrodes 102 are located on any exposed surface of the housing 104, such that both the anode and cathode are exposed to the healing site, and electrically isolated from the pizeo unit.

Because of the nature of the piezogenerator 106 as a power source, the electrical stimulation is inherently in sync with an anatomical mechanical load applied, and the amount/intensity of the stimulation is a function of the anatomical load amplitude and frequency and the electrical resistance between the electrodes 102. In the preferred embodiment, the piezogenerator 106 converts dynamic load to an AC electrical signal, which is passed through the impedance matched rectifying circuit 108 to generate a pulsed negative DC electrical signal that is passed through internal electrical connections to the electrodes 102. In other embodiments, the piezogenerator 106 as the incorporated power source is replaced with a battery activated by a force sensor in line with circuitry or other electrical components that provide negative DC pulsed electrical stimulation in sync with the force on the implant assembly 100 due to the changes in anatomical load.

The implant assembly 100 includes at least two external electrodes 102 that may be configured as anodes or cathodes and must be exposed to a healing site. The housing 104 is disposed within the healing site and incorporates the piezogenerator 106 and the circuit 108. In some embodiments, the piezogenerator 106 and circuit 108 are disposed within the housing 104 such that there is an open cavity (i.e., graft window) that extends from the superior surface of the implant 102 to the inferior surface of the implant 102. In other embodiments, the housing 104 includes an additively manufactured or 3D printed material such as, for example, titanium, titanium alloy, or a polymer such as polyether ether ketone (PEEK). The housing 104 can include one or more components that enable an insertion and assembly of the encapsulated piezogenerator 106 and circuit 108. In other embodiments, the housing may be a standardized cartridge that is assembled with other implant components to make up the general shape of a spinal implant. In other embodiments, the other implant components could be 3D printed with materials such as titanium, titanium alloy, or a polymer such PEEK which contain an organic or porous lattice type structure. The other components could serve a structural purpose and or could serve as an external electrode or antenna in the event they are electrically conductive.

In another embodiment, two or more housing 104 components constructed of additively manufactured material with a matching form fitting cavity around the piezogenerator 106 and circuit 108 are assembled together and bonded around an open cavity in order to hermetically seal the piezogenerator 106 and circuit 108. The bonding can be in the form of a perimeter laser weld operation or medical grade epoxy or polymer molding operation. The piezogenerator 106 and circuit 108 may be pre-encapsulated with epoxy or other polymer material prior to assembly into a secondary housing 104 or it may be encapsulated after assembly into the housing 104.

In another embodiment, the electrode or electrodes 102 include an additively manufactured or three dimensional (3D) printed conductive material, such as titanium or titanium alloy, that surrounds the housing 104, piezogenerator 106, and circuit 108. The electrodes 102 may be one or more components that enable the insertion and/or assembly of the piezogenerator 106, circuit 108, and housing 104.

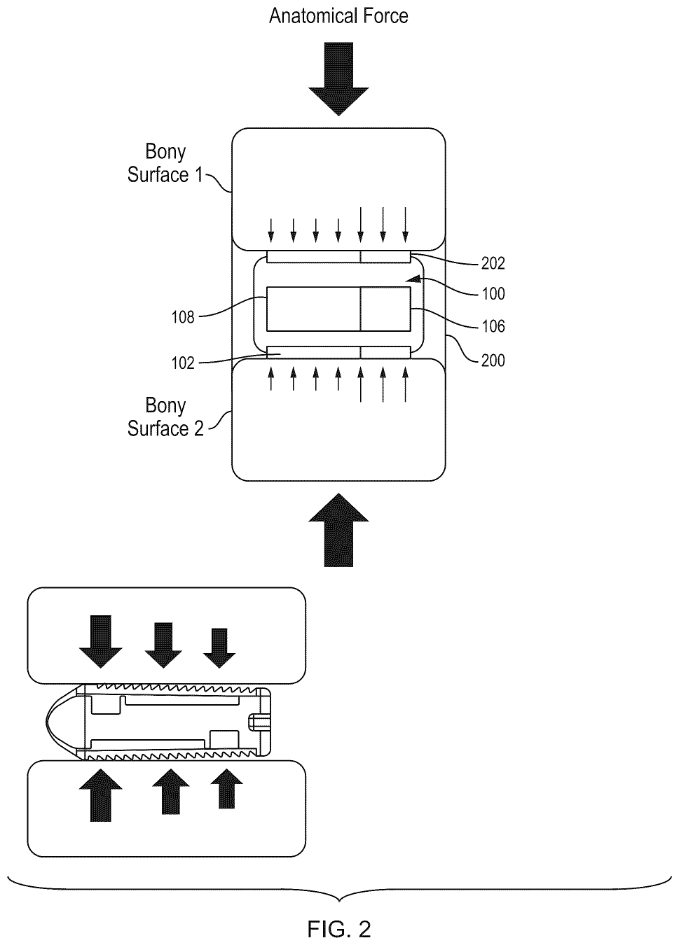

FIG. 2 illustrates the implant 100 in the load path and graphically demonstrates load distribution to the implant 100. More specifically, the piezogenerator 106 and the circuit 108 assembly are aligned in the implant housing 104 such that the assembly 100 is in line with the anatomical load path between a bony surface 1 and a bony surface 2. In the preferred embodiment, the piezogenerator 106/circuit 108 assembly is hermetically sealed and bonded within the implant housing 104 and external electrodes 102. In other embodiments, the housing 104 is injection molded in and around the piezogenerator 106/circuit assembly 108. In a preferred embodiment, the electrodes 102 are integrated/bonded with the implant assembly 100 and include a force focus area 202 that directs an anatomical force directly over the piezogenerator 106. In other embodiments, the force focus area 202 is directed over a force sensor. In one embodiment, the force focus area 202 of the electrode 102 is a geometrical feature of the external surface component. In other embodiments, the force focus area 202 is a material property change, creating a stiffer area of the electrode 102 to focus the force over the piezogenerator 106. In still other embodiments, the force focus area 202 is additively manufactured or 3D printed material such as titanium, titanium alloy, or a polymer such as PEEK, where the structure of the material is designed to be porous or have variable stiffness throughout the overall structure and directed over the piezoelectric material and such that certain areas of the structure have a higher or lower stiffness than other areas of the structure in order to produce the force focus area 202. In other embodiments, the structure of the additively manufactured structure has self-imposed physical limits of displacement that would enable the force to be delivered first to force focus area 202 and second to the rest of the surface area.

The implant assembly 100 is inserted in between two bony surfaces 1, 2, creating a healing site 200 between bony surface 1 and bony surface 2 in which mechanically synced electrical stimulation is delivered and load is measured. As the mechanically synced electrical stimulation is delivered to the healing site 200, bone growth is stimulated. As the bone grows in and around the implant assembly 100, the anatomical force is distributed across the healing site 200 as the new bone growth carries more load and decreases the load carried by the implant assembly 100. The MSES is generated by the piezogenerator 106 and delivered to the healing site by the electrical connection of the electrodes 102 with the circuit 108. When the share of the anatomical load reaches a certain threshold, the piezogenerator 106 no longer generates an electrical signal, thus the MSES delivered to the healing site 200 ceases. Endogenous healing cycles mimic this same process, the bone healing process is activated until new bone is formed, and then the cycle triggers are inactive until new bone growth is necessary to transfer load appropriately.

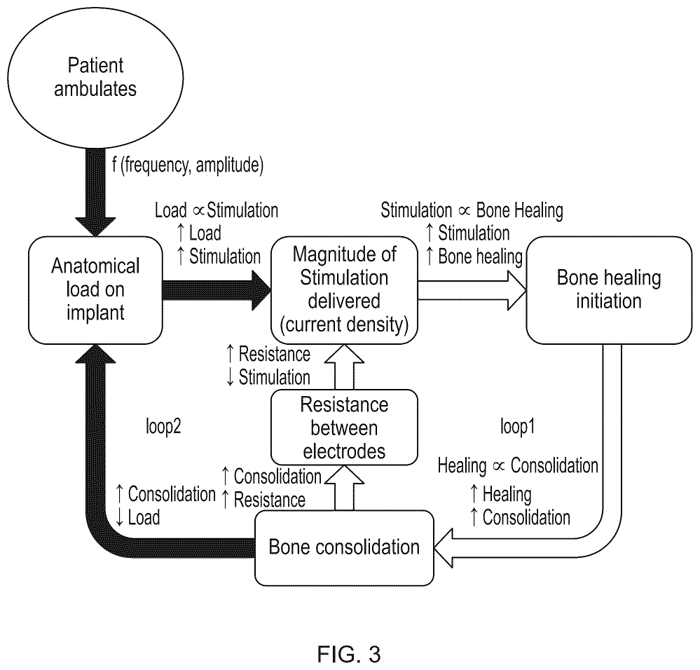

The human powered smart implant 100 of the present invention utilizes the piezogenerator 106 and the circuit 108 integrated within the implant assembly 100 to deliver mechanically synced electrical stimulation to a healing site between adjacent bony surfaces and measure load and other physiologic indicators. FIG. 3 depicts the electrical stimulation regulation through a biological feedback loop 300, which is inherently stable due to the negative feedback loop of anatomical force on the implant assembly 100 and healing progression at the healing site.

When implanted between two bony surfaces, the implant assembly 100 undergoes dynamic load due to the anatomical force generated during patient ambulation. The components of that dynamic anatomical force, namely amplitude and frequency, are a function of the location of the implant 100 and patient characteristics (e.g., body mass index). Because the electrodes 102 are designed with a force focus area 202 directly above the piezogenerator 106, the stimulation delivered to the healing site is proportional to the load applied to the implant 100. Similarly, as the stimulation is delivered through the electrodes to the healing site, bone healing is initiated by the electrical stimulation. As healing progresses and consolidation occurs, the electrical resistance between the electrodes increases, which decreases the magnitude of stimulation delivered to the healing site. This loop1 continues until the bone healing consolidation reaches the next phase (loop2), where the anatomical load share transferred to the implant assembly 100 decreases as the healing fusion mass carries more of the load. As consolidation of the fusion mass increases, the load on the implant decreases, which then decreases the magnitude of stimulation delivered. This then starts the process in loop1 again. Using a load activated power source (i.e., the piezogenerator 106) enables the biological negative feedback loop to regulate the electrical stimulation delivered as a function of the healing progression.

It should be noted that while FIG. 3 illustrates a force induced stimulation, other sustained alternative power sources may be employed.

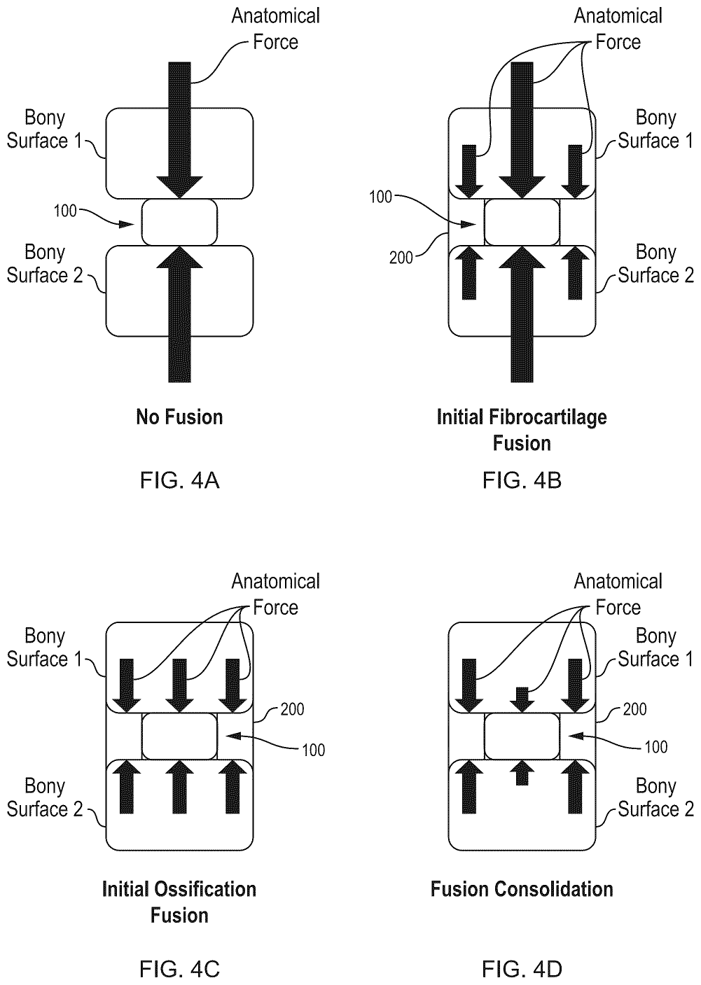

In FIGS. 4A-4D, a general concept of how implant load relates to the stages of bone healing within the healing site 200 and anatomical force are shown. Immediately after surgery, it can initially be assumed that all the anatomical force acting on bony surface 1 during ambulation passes through the implant assembly 100, as depicted in FIG. 4A. As healing progresses at the healing site 200, a bony fusion begins to form between the adjacent bony surface 1 and bony surface 2. At the initial stages of healing, as depicted in FIG. 4B, the fusion mass in the healing site 200 is low in stiffness, as there is fibrocartilage formation and no consolidation yet of the bone graft. While the fibrocartilage is relatively low in stiffness, it can carry some of the force/load away from the implant assembly 100, as depicted by the distribution of arrows. Over time, the fusion mass in the healing site 200 starts to consolidate, and the fibrocartilage begins to ossify, depicting an initial ossification fusion as shown in FIG. 4C. The healing bone across the healing site 200 increases in stiffness due to calcification, resulting in less force being carried by the implant assembly 100 and more load carried by the bone forming between bony surface 1 and bony surface 2. In the final stages of healing, if there is complete consolidation of the bone fusion mass and formation of structurally sound, ossified bone across an interbody gap healing site 200, the fusion mass then carries most of the force, leaving very little force carried by the implant assembly 100 as shown in FIG. 4D. Over time, as the bony fusion mass across the interbody space increases in volume and/or quality, the decreasing load on the implant assembly will decrease the amount of MSES delivered by the implant assembly to the healing site 200, and eventually the load amplitude will be too low to generate any electrical signal from the integrated power source. In addition to the change in the mechanical load environment, the electrical resistance/impedance of the healing bone will change as it matures and calcifies. The resistance will increase as more bone consolidation occurs, decreasing the stimulation delivered. It should be noted that the time for the fusion process to go through the various stages is variable for each patient, but the negative feedback loop that integrates into the natural healing biological loop ensures an inherently stable system that mimics Wolff's Law, as was shown in FIG. 3.

In the preferred embodiment, the piezogenerator 106 functions as a power generator, transducing the anatomical load on the implant to mechanically synced electrical stimulation delivered to the healing site 200 through the electrodes 102 and transducing the anatomical load on the implant to power the microcontroller and/or charge an on board solid state battery. In other embodiments, the piezogenerator 106 and circuit 108 are configured to function as a load sensor. The circuit 108 is configured to collect, store, transmit the load experienced by the piezogenerator 106 through the implant assembly 100 within the healing site 200 and transmit the changes over time. The changes in the load experienced by the implant assembly 100 over time may indicate healing progression—as the load on the piezogenerator 106 decreases, this may indicate bone growth and healing in the healing site 200. This data can be used to track healing.

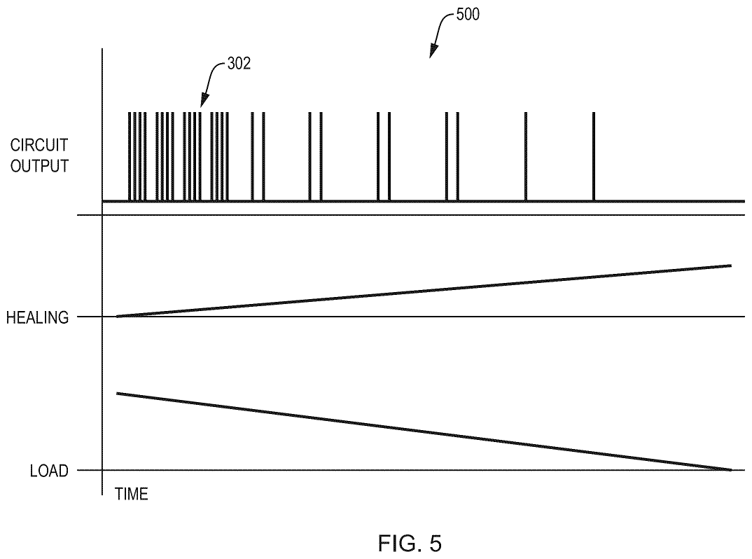

FIG. 5 is an exemplary time series graph 500 of the electrical output of the preferred embodiment in relation to the healing progression over time and the mean anatomical load applied to the implant. These relationships represent the same relationships described above and illustrated in FIGS. 4A, 4B, 4C and 4D. The nature of the pulsed mechanically synced electrical output is shown in the top time series. In the preferred embodiment, the electrical output is a monophasic, square pulsed voltage capped signal. The maximum voltage threshold is designed to be within safe physiological limits for electrical stimulation. As the load on the implant decreases as healing progresses, the number of pulses (duty cycle) delivered for a given mechanical load cycle (e.g., each step during ambulation) decreases. The pulse count is a function of the dynamic load amplitude and frequency applied to the implant during patient motion. The pulse width of individual pulses is a function of the resistance between the anode and cathode, and will increase as healing progresses and the tissue resistance of the healing site increases as bone calcifies. This will decrease the overall energy delivered to the healing site over time as healing progresses. The implant 100 will enter a non-active phase (e.g. no pulsed electrical output) when the load amplitude or electrical resistance decreases beyond the designed thresholds. In another embodiment, the electrical output is a bi-phasic pulsed wave. In another embodiment, the electrical output is an alternating sine wave. In another embodiment, the electrical output is a constant positive or negative signal. In yet another embodiment, the electrical signal is a ramp signal.

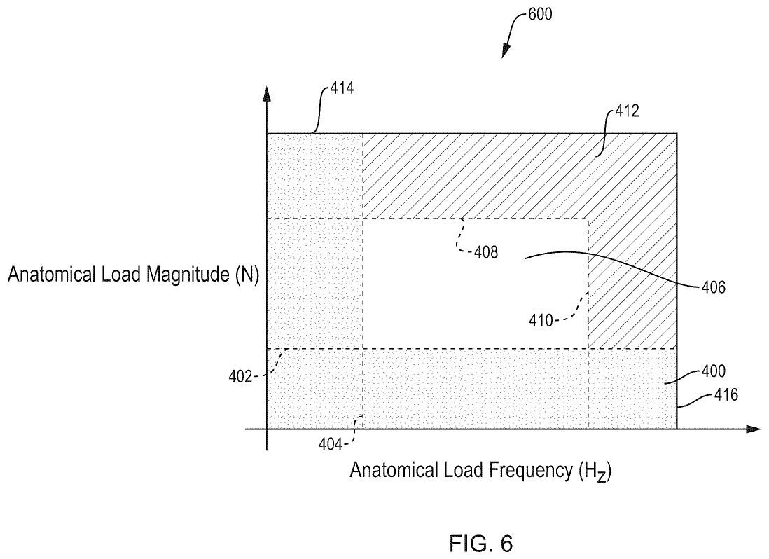

FIG. 6 depicts exemplary electrical operation regions of the implant 100 within physiological limits, creating built-in control loops within the device 100. The plot 600 shows the nature of the mechanically synced electrical stimulation as a function of the anatomical load magnitude and anatomical load frequency experienced by the implant assembly as the patient ambulates. Under certain conditions (low load magnitude and low load frequency) the electrical stimulation will be inactive, meaning the implant 100 is in a stimulation disabled state 400 and acts as a standard implant stabilizing the healing site. Once the loading condition changes such that the anatomical load or the anatomical frequency surpasses and sustains a pulsed stimulation load threshold 402 or a pulsed stimulation frequency threshold 404, the delivered electrical stimulation is pulsed in nature. Under expected physiological and anatomical force amplitudes and frequencies of typical patient populations, the implant 100 remains in a pulsed stimulation active state 406. The mechanically synced electrical stimulation that is delivered is a non-constant, pulsed negative DC signal in nature with a defined voltage threshold. The pulsed stimulation active state 406 is expected under all activities of daily living (e.g., walking, sitting, standing, and so forth) that create dynamic anatomical force conditions that fall within the thresholds defined.

Although rare under typical physiological limits (>1000N amplitude loads at a sustained frequency >4 Hz), there exists a constant stimulation load threshold 408 and constant stimulation frequency threshold 410 that causes the delivered electrical stimulation to be constant instead of pulsed. During the constant stimulation active state 412, the current delivered will be constant instead of pulsed, but remains within safe limits, as the voltage is capped at 4V. This state is only active if the load experienced by the implant is high enough to surpass the constant stimulation load threshold 408 and sustained or the load frequency is higher than the constant stimulation frequency threshold 410 and sustained. The anatomical load threshold 414 would only be surpassed beyond mechanical limits of the implant assembly 100 and/or crush force of bone in which the implant assembly 100 would be damaged and thus no stimulation delivered. Similarly, the anatomical frequency threshold 416 represents the threshold above which it would not be probable to surpass and sustain this frequency under any physiologic conditions.

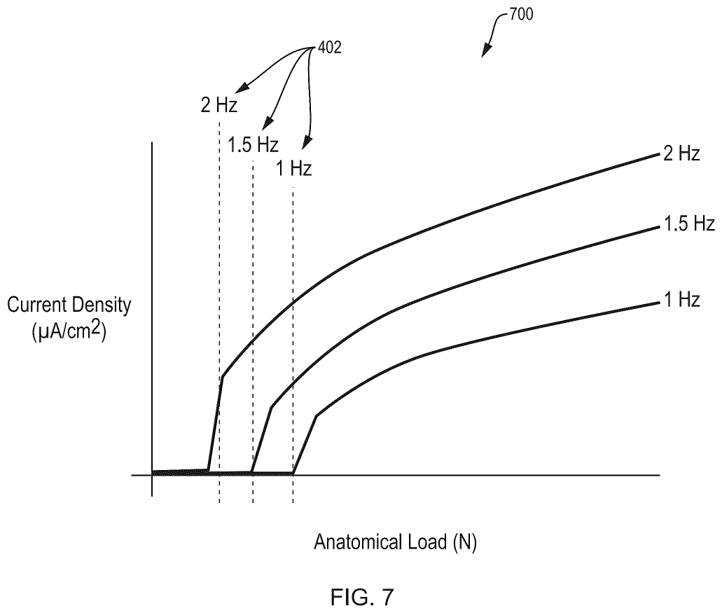

FIG. 7 is an exemplary graph 700 that represents the relationship between current density and anatomical load experienced by the implant 100 at typical frequencies of everyday activities (e.g., patient ambulation). Specifically, at a given stage of healing in and around the healing site 200 the resistance will be fairly constant, meaning that the amount of electrical stimulation delivered is a function of the load amplitude and frequency experienced by the implant assembly 100. As the frequency increases, the current density increases. For each frequency, the pulsed stimulation load threshold 402 represents the lowest anatomical load that activates the pulsed nature of the electrical stimulation. The pulsed stimulation load threshold 402 decreases as the frequency of the load increases. For example, if a patient takes more steps per second, the load necessary to “turn on” the pulsed stimulation state of the implant 100 will be lower.

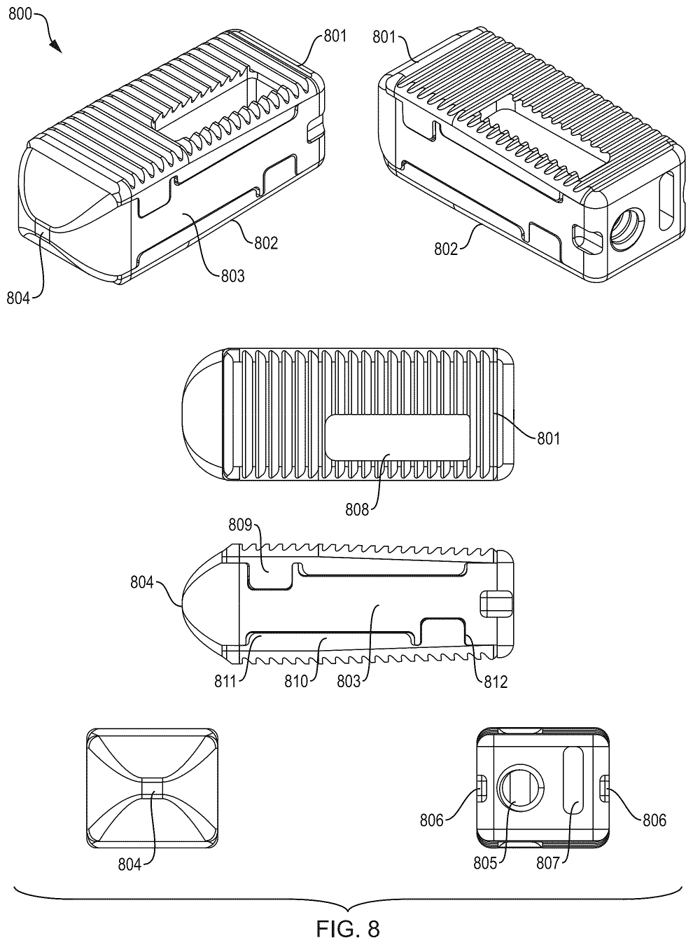

FIG. 8 represents an exemplary assembled implant 800 in a preferred embodiment. The assembly 800 includes a housing 803 that is mechanically fixed via a clip and rail mechanism to a top electrode endplate 801 and a bottom electrode endplate 802. In the preferred embodiment, the housing 803 is made of a biocompatible polymer (e.g., PEEK) and the endplate electrodes 801, 802 are machined or 3D printed from biocompatible titanium. In other embodiments, the materials are changed to create a different set of mechanical properties conducive to bone growth during healing. In the preferred embodiment, the housing 803 includes design features such as a bullet nose 804, an inserter threaded connection 805, inserter stabilizer 806 features, a potting access 807 feature and the associated rail recess 811 and clip recess 812. The implant 800 is inserted into the healing site 200 utilizing a tool that threads into the inserter threaded connection 805 and clips into the inserter stabilizer recesses 806 to facilitate a firm grip on the implant 800. Once inserted and properly placed in the healing site 200, the inserter 800 is unthreaded and removed from the assembled implant 800. The top electrode endplate 801 and bottom electrode endplate 802 include the endplate clip 809 and endplate rail 810 that fit into the associated recesses 811, 812 in the housing 803 to mechanically lock the endplates to the housing. In the preferred embodiment, both endplates have design features (e.g., teeth) to that interface with the adjacent bony surfaces to promote implant stabilization and limited motion within the healing site 200. In the preferred embodiment, the endplates and housing are designed to include an opening that is disposed between the top and bottom surfaces, a graft window 808 allowing tissue healing to occur between the adjacent bony surfaces. The implant assembly 800 represents the external, tissue contacting components of the implant.

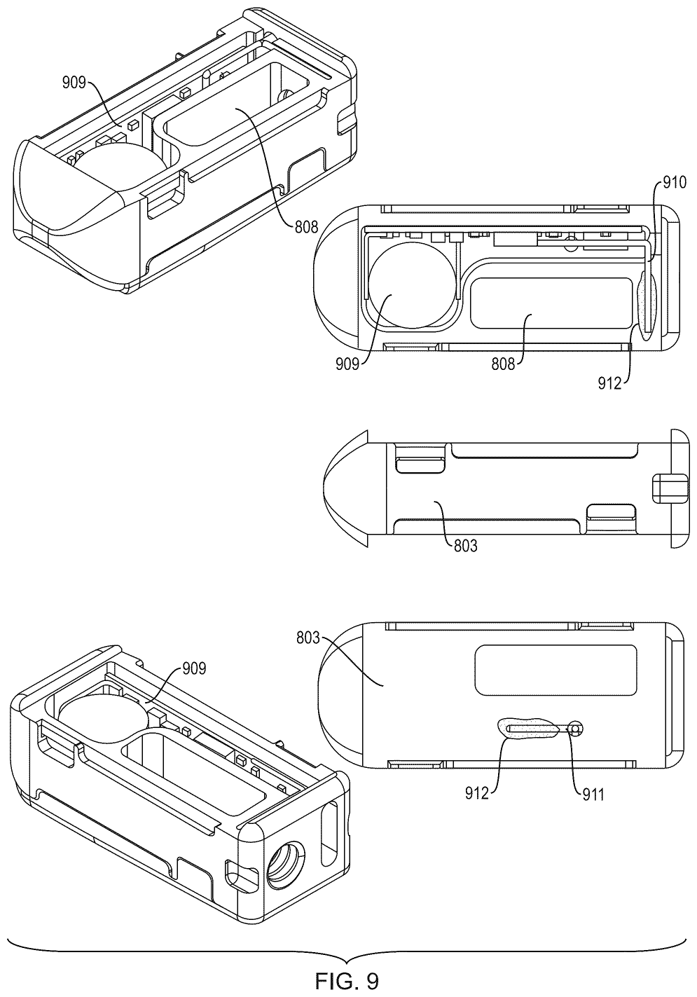

Within the implant assembly 800 the internal components of the preferred embodiment are represented in FIG. 9. Inside the housing 803, the circuit piezo sub-assembly 909 is disposed between the endplate electrodes. In the preferred embodiment, the components of the circuit piezo sub-assembly 909 are encapsulated within the housing. The negative output lead 910 is electrically connected to the top endplate electrode 801 via conductive epoxy 912. Similarly, the positive output lead 911 is electrically connected to the bottom endplate electrode 802 via conductive epoxy 912.

In embodiments, the present invention may include Posterior Lumbar Interbody Fusion (PLIF), Anterior Lumbar Interbody Fusion (ALIF) and Oblique Lateral Lumbar Interbody Fusion (OLIF) cage designs with a modular insert.

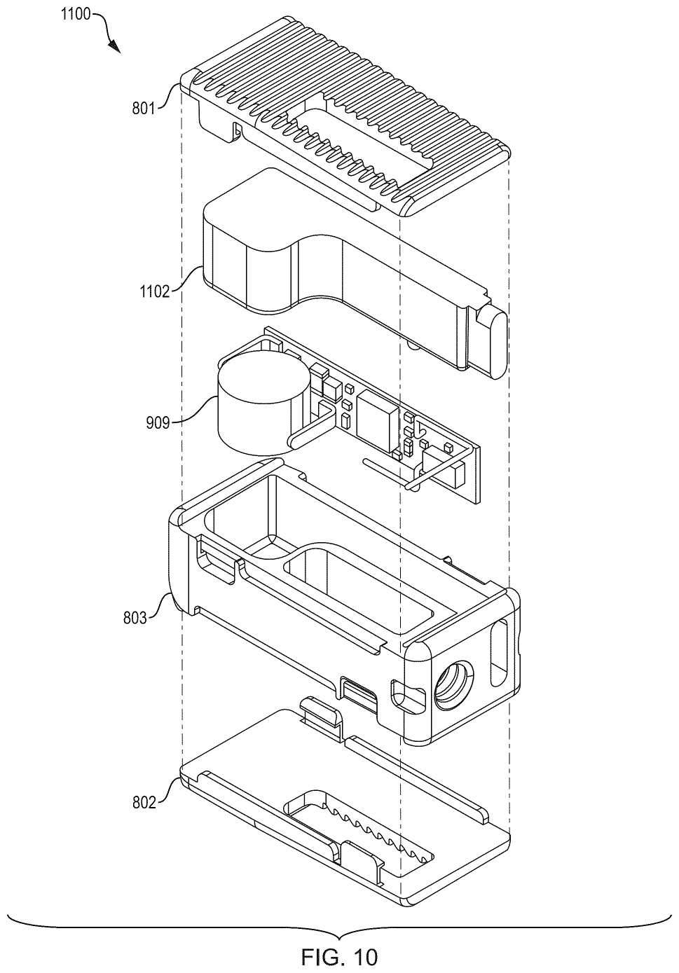

FIG. 10 presents an exemplary exploded implant assembly 1100. As discussed above, once assembled, the circuit piezo sub-assembly 909 is potted in the implant housing 803. The formed potting epoxy 1102 electrically insulates the electrical components on the circuit and encapsulates the components from ingress of fluid once in the healing site 200. The assembly process includes clipping the endplate electrodes to the housing and then potting the entire assembly through the potting access 807, creating a singular mechanically bonded implant assembly 800. In the preferred embodiment the potting epoxy 1102 is a two-part medical grade translucent epoxy with similar mechanical properties to the housing 803. In other embodiments, the housing and formed potted epoxy can injection molded or formed with other methods.

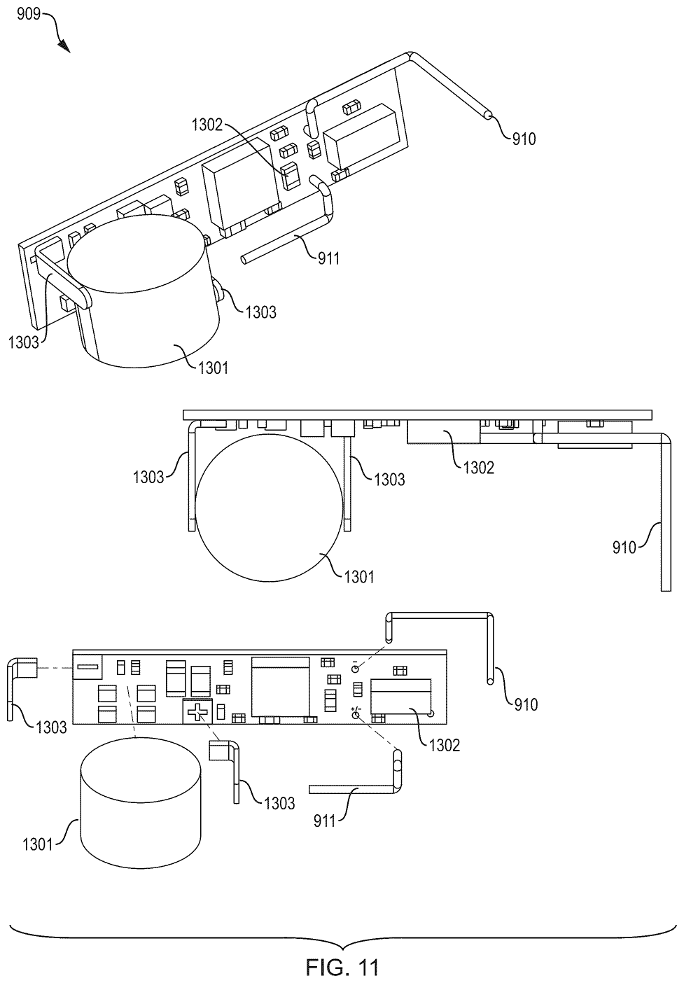

FIG. 11 presents an exemplary diagram of the components within the circuit piezo sub-assembly 909. The shims 1303 electrically bond the piezogenerator 1301 to the circuit 1302. In the preferred embodiment, the shims 1303 are electrically bonded to the positive and negative terminals of the piezogenerator 1301 via conductive epoxy. The shims 1303 are then soldered to the circuit 1302. The pulsed mechanically synced electrical output 302 of the circuit 1302 is conducted via the negative electrode lead 910 connected to the top endplate electrode 801 and the positive electrode lead 911 is connected to the bottom endplate electrode 802.

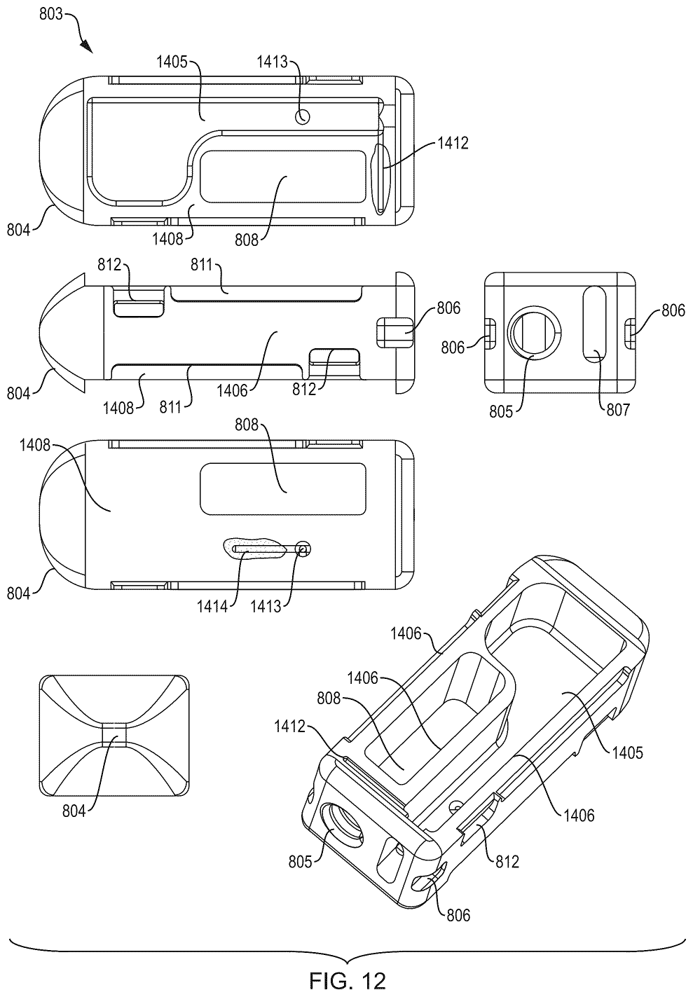

FIG. 12 demonstrates exemplary features of the housing 803. These features are specific to the preferred embodiment but should not be understood as the only features covered in the present invention. The clip recess 812 and rail recess 813 are incorporated into the side walls 1406 of the housing 803. The size and alignment of the recesses match the corresponding clip and rail features on the electrode endplates. The circuit piezo pot 1405 houses the circuit piezo assembly 909. On the bottom surface of the circuit piezo pot 1405 there is a positive lead through hole 1413. The hole 1413 is positioned to align with the positive electrode lead 911. On the bottom surface 1408, connected to the positive lead through hole 1413 is a positive lad channel 1414 in which the positive electrode lead 911 sits in. On the top surface 1408 of the housing 803 there is a negative lead channel 1412 in which the negative electrode lead 910 sits in. Conductive epoxy 912 is spread across both lead channels before the corresponding endplate electrode 801, 802 is clipped on to the housing 803, creating an electrical and mechanical bond, respectively.

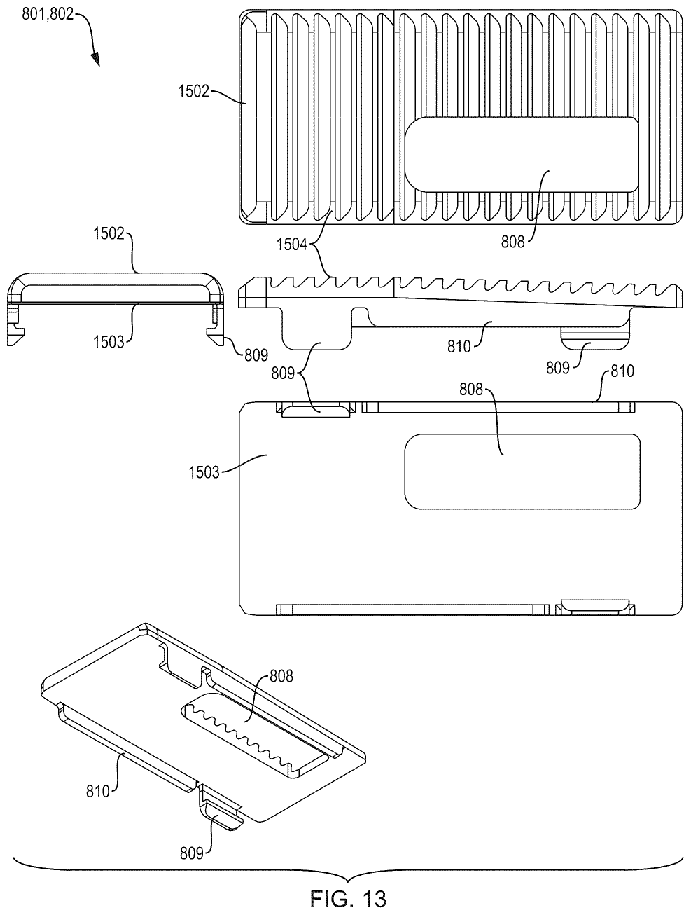

FIG. 13 illustrates exemplary features of the electrode endplates 801, 802. These features are specific to the preferred embodiment but should not be understood as the only features covered in this invention. The opening for the graft window 808 is biased toward the side in which the graft window is located in the housing 803 on either the top or bottom surface correspondingly. The outer surface 1502 includes anti-migration teeth 1501. The specific design features of the outer surface 1502 and anti-migration teeth 1501 can be altered based on the intended anatomical location of the healing site 200 and respective needs to ensure the assembled implant 800 stays in place once implanted. In the preferred embodiment, the force focus feature 1504 is a portion of the endplates that is perpendicular to the axis of anatomical load. This portion of the endplate ensures that the load is normal to the top face of the piezogenerator 1301. The inner surface 1503 of the top electrode endplate 801 is electrically bonded to the negative electrode lead 910 and mechanically bonded to the entire assembly via the formed potting epoxy 1102 and endplate clip 809 and endplate rail 810 features. The inner surface 1503 of the bottom electrode endplate 802 is electrically bonded to the positive electrode lead 911 and mechanically bonded to the entire assembly via endplate clip 809 and endplate rail 810 features.

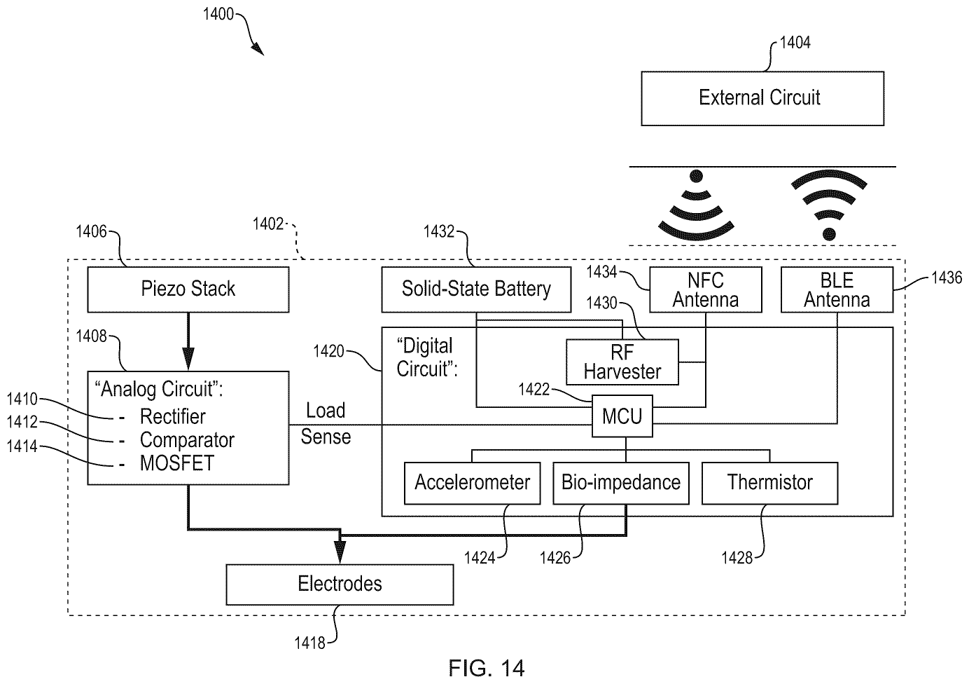

In FIG. 14, a block diagram of an exemplary system 1400 in accordance with the present invention is shown. The system 1400 includes an implant 1402 and an external circuit 1404.

The implant 1402 includes a piezo construct 1406 linked to an analog circuit 1408. The analog circuit 1408 includes, for example a rectifier 1410, a comparator 1412 and a metal-oxide-semiconductor-field-effect transistor (MOSFET) 1414.

The analog circuit 1408 is linked to electrodes 1418 and to a digital circuit 1420. The digital circuit 1420 includes, for example, microcontroller unit (MCU) 1422. An accelerometer 1424, a bioimpedance circuit 1426, a thermistor (i.e., resistance thermometer) 1428 and a radio frequency (RF) harvester 1430 are lined to the MCU 1422. The electrodes 1418 are linked to the bioimpedance circuit 1426, which is configured to estimate a body composition.

The implant 1402 includes a solid-state battery 1432 linked to the MCU 1422 and RF harvester 1430.

The implant includes a near field communication (NFC) antenna 1432 and a Bluetooth Low Energy (BLE) antenna 1436. The NFC antenna 1434 is linked to the MCU 1422 and RF harvester 1430. The BLE antenna 1436 is linked to the MCU 1422. Both the NFC antenna 1434 and the BLE antenna 1436 are configured to provide wireless communication between the implant 1402 and the external circuit 1404.

In the foregoing description, it will be readily appreciated by those skilled in the art that modifications may be made to the invention without departing from the concepts disclosed herein. Such modifications are to be considered as included in the following claims, unless the claims by their language expressly state otherwise.

What is claimed is:

1. An implant assembly comprising:
a housing configured to store components that generate biomechanically activated electrical energy;
a first electrode positioned on an external surface of the housing; and
a second electrode positioned on an external surface of the housing and isolated from the first electrode,
the components comprising:
a force activated piezogenerator electrically connected to an impedance matched rectifying circuit and digital microcontroller,
wherein the piezogenerator transduces a mechanical load to an electrical signal that is passed through the impedance matched rectifying circuit and delivered through the first and second electrode to a healing site when an anatomical force is applied to the implant surfaces that make contact with two bony surfaces.

2. The implant assembly of claim 1 wherein the piezogenerator converts dynamic load to an alternating current (AC) electrical signal, which is passed through the impedance matched rectifying circuit to generate a pulsed negative direct current (DC) electrical signal that is passed through internal electrical connections to the first and second electrode.

3. The implant assembly of claim 2 wherein the piezogenerator and the impedance matched rectifying circuit are disposed within the housing such that there is an open cavity that extends from the second electrode to the first electrode.

4. A human powered implantable device comprising:
a first electrode positioned on an exterior surface of a housing;
a second electrode positioned on an exterior surface of the housing and isolated from the first electrode;
a microcontroller; and
piezoelectric materials within the housing to generate load-induced power and run a measurement and storage function, the power configured to generate electrical stimulation to promote bone growth and support data collection related to load, impedance, acceleration, temperature, and other physiologic signals.

5. The human powered implantable device of claim 4 wherein the generated power delivers stimulation via mechanically synced electrical stimulation (MSES).

6. The human powered implantable device of claim 5 wherein the generated power activates measurement and storage function to collect and store data during discrete periods of time for later transmission to an external data acquisition system.

7. The human powered implantable device of claim 6 wherein the data is selected from one or more of AC voltage from the piezoelectric materials, local temperature from the microcontroller, impedance between first and second electrode, and acceleration for gait analysis.

8. The human powered implantable device of claim 7 further comprising an external device.

9. The human powered implantable device of claim 8 wherein the data is transmitted to the external device.

10. The human powered implantable device of claim 9 wherein the external device is configured to monitor trends of activity.

11. The human powered implantable device of claim 10 wherein the trends comprise prediction of post-operative fusion mass formation, device loosening, and biological activity to indicate infection and trending.

12. The human powered implantable device of claim 11 wherein the external device is a smartphone.

13. The human powered implantable device of claim 12 wherein the smartphone includes an app configured to monitor implant power and control a switch in the implant to toggle between high and low power sensing mode.

14. The human powered implantable device of claim 13 wherein the data is leveraged with a database from a Electronic Medical Record, preclinical and clinical product studies to process the data and correlate clinical outcomes in order to inform changes to treatment guidelines and current standard of care.

15. A system comprising:
an external circuit; and
an implant, the implant configured to wirelessly communicate with the external circuit, the implant comprising:
a piezo construct;
an analog circuit, the piezo construct linked to the analog circuit;
a digital circuit;
electrodes, the electrodes linked to the analog circuit and the digital circuit;
a solid state battery, the solid state battery linked to the digital circuit;
a near field communication (NFC) antenna; and
a Bluetooth Low Energy (BLE) antenna, the NFC antenna and the BLE antenna linked to the digital circuit.

16. The system of claim 15 wherein the analog circuit comprises:
a rectifier;
a comparator; and
a metal-oxide-semiconductor-field-effect transistor (MOSFET).

17. The system of claim 16 wherein the digital circuit comprises a microcontroller unit (MCU).

18. The system of claim 17 wherein the digital circuit further comprises:
a radio frequency (RF) harvester;
an accelerometer;
a bio-impedance circuit; and
a thermistor.

19. The system of claim 18 wherein the MCU is linked to the analog circuit, the radio frequency (RF) harvester, the accelerometer, the bio-impedance circuit, the thermistor, the NFC antenna and the BLE antenna.
